(19) United States
(12) Patent Application Publication
(10) Pub. No.: US 2024/0016760 A1
(43) Pub. Date: Jan. 18, 2024

(54) Multivitamin

(71) Applicant: Michael Walters, Huntsville, AL (US)

(72) Inventor: Michael Walters, Huntsville, AL (US)

(21) Appl. No.: 18/198,471

(22) Filed: May 17, 2023

Related U.S. Application Data
(63) Continuation-in-part of application No. PCT/US22/37053, filed on Jul. 14, 2022.

(51) Int. Cl. A61K 31/015; A61K 31/07; A61K 31/355; A61K 31/122; A61K 31/375; A61K 36/534; A61K 33/30; A61K 45/06

(52) U.S. Cl. CPC A61K 31/015; A61K 31/07; A61K 31/355; A61K 31/122; A61K 31/375; A61K 36/534; A61K 33/30; A61K 45/06

(57) ABSTRACT

The present disclosure provides a multivitamin specifically formulated with an amended non-staining beta-carotene, or pro-vitamin A. In another aspect, the present disclosure provides a multivitamin for patients undergoing CFTR modulator therapy. The multivitamin includes, in various embodiments, pro-vitamin A, vitamin E, a solubilizer, a bile fluid secretion stimulator, vitamin C and a probiotic formulation.

### CROSS-REFERENCE TO RELATED APPLICATIONS

This application claims priority to, and the benefit of, pending Patent Cooperation Treaty (“PCT”) Application No. PCT/US22/37053 filed Jul. 14, 2022, which in turn claimed priority to, and the benefit of, U.S. Provisional Patent Application No. 63/233,874 filed Aug. 17, 2021; 63/282,259 filed Nov. 23, 2021; 63/318,864 filed Mar. 11, 2022, and 63/318,928 filed Mar. 11, 2022.

### FIELD OF THE DISCLOSURE

The present disclosure pertains to the field of nutritional supplements such as vitamin A and multivitamins containing vitamin A. More specifically, the present disclosure pertains to a beta-carotene containing multivitamin and vitamin A where the beta-carotene component has been amended to eliminate the discoloring and staining aspects commonly associated with beta-carotene. Additionally, the present disclosure pertains an improved multivitamin for those suffering from Cystic Fibrosis (“CF”) and that are undergoing CF transmembrane conductance regulator gene (“CFTR Gene”) modulator formulation (“MF”) therapy.

### BACKGROUND

Beta-carotene is a red-orange pigment found in plants that gives them their color. It gives yellow and orange fruits and vegetables their rich hues. Beta-carotene, sometimes referred to as pro-vitamin A, is a precursor to retinol, the active form of vitamin A. Beta-carotene discolors and stains, and often stains irreversibly. Beta-carotene is recognized as a safe source for vitamin A, as beta-carotene is only absorbed and converted to the active form of vitamin A as needed by the body. Hence the name pro-vitamin A. The most common side effect of excessive beta-carotene intake is a physically harmless condition that results in orange skin discoloration or hue due to the deposition of the carotenoid in the epidermis of the skin. This is in stark contrast to the over intake of preformed vitamin A, which can cause serious adverse conditions.

Vitamin A is an essential nutrient to the body. Vitamin A deficiency can cause symptoms such as fatigue, skin issues, a weakened immune system, and night blindness. Vitamin A also has antioxidant and anti-inflammatory properties to help protect the body's cells from damage. Most people in the U.S. get enough vitamin A from their diets. However, people with certain conditions are likely to have a vitamin A deficiency (e.g., those with digestive disorders). Conditions affecting pancreatic function can result in digestive disorders, both those which are genetically based (e.g., Cystic Fibrosis) and those due to non-genetic causes (e.g., Crohn's, malabsorption secondary to alcoholism, diabetes, etc.), and often require vitamin A supplementation.

Many multivitamin formulations used in cystic fibrosis (“CF”) care include both preformed vitamin A and pro-vitamin A (i.e., beta-carotene); these formulations and formulations that represent current prior art, however, introduced a significant issue—discoloration and staining—due to the beta-carotene. Materials (e.g., clothing, carpeting with liquid forms), the oral cavity (e.g., teeth and tongue with chewable tablets), and fingers and hands (e.g., with liquid and softgel forms) are subject to discoloration and staining by beta-carotene. Concerns around discoloration and staining with prior art liquid multivitamins, whether due to the infant or toddler spitting out the liquid, or a spill, often results in the infant or toddler being administered the liquid prior art beta-carotene containing multivitamin in the bathtub. Absorption of fat-soluble vitamins, however, requires the presence of fats; and few parents provide the needed fat intake when liquid beta-carotene multivitamins are administered in a bathtub. This can negatively affect absorption and reduce the clinical benefit and treatment goals of supplementation.

Prior art beta-carotene chewable tablets also present a problem. As the tablet is chewed and broken into small pieces to enable swallowing, the beta-carotene containing small particles can adhere to the teeth, tongue other mucosal surfaces and cause discoloration. This is particularly a concern for individuals wearing braces, where small pieces of the chewable tablet can lodge between the teeth and apparatus; and if one's teeth are not immediately brushed following the chewing of a beta-carotene containing multivitamin, staining can present an issue over time.

Staining of fingers and hands also occurs when the gelatin softgel seal, which runs longitudinally along the softgel (i.e, along the y, versus x axis) does not fully seal, or later separates. Heat, cold, and physical agitation can cause separation of this seal and leakage of the softgel content. When this occurs post bottling, these leaking softgels are called “latent leakers”, and the content from the “latent leakers” can transfer to the outside of other softgels, and to the sides and bottom of the container. The container may include the original packaging (e.g., bottle), or a weekly or daily pill case, when used. It is important to note that a single bottle of softgels can have no, one, or multiple “latent leakers”. This issue was so significant, that Allergan withdrew their AquADEK® Softgel Multivitamins from the U.S. market several years ago. AquADEK was the #1 softgel used in CF. With just one latent leaker, the beta-carotene containing content leaks along the adulterated seal line, transferring the orange-red multivitamin matrix to the outside of other softgels and to the interior of the bottle or pill box. When a softgel serving is removed for administration, the leaked content—now on the surface of other softgels or container—is transferred. This transfer of the leaked orange-red content causes discoloration and staining of fingers, hands, or both.

Today, Poly-Vi-Sol® infant liquid multivitamins command a significant share of the over the counter (“OTC”), infant, liquid multivitamin market. While Poly-Vi-Sol is non staining, this aspect is due to the absence of pro-vitamin A (i.e., beta-carotene) in the formulation, and reliance of the formulation on 100% preformed vitamin A. Like found in CF, preformed vitamin A is insatiably absorbed in the general population and presents the same risks, as any preformed vitamin A ingested is absorbed by the body, whether required by the body, or not. This presents a potential risk for toxicity (i.e., hypervitaminosis A), based on dietary intake of vitamin A and supplemental of vitamin A from a serving of Poly-Vi-Sol—100% preformed vitamin A. This risk may be mitigated by replacing the preformed vitamin A in Poly-Vi-Sol with pro-vitamin A (i.e., beta-carotene) to support personalized and targeted serum retinol levels based on the body's biofeedback systems. Using a non-discoloring, non-staining, beta-carotene addresses the limitation of the current pro-vitamin A, a limitation that has likely driven the use of preformed vitamin A, despite its documented risks.

Cystic Fibrosis (“CF”) is a complex multi-organ disease caused by mutations in the CFTR Gene. The protein encoded by the CFTR Gene, the CFTR Protein, forms a cAMP-regulated ion channel responsible for chloride and bicarbonate secretion in epithelial cells. The CFTR Protein also regulates the epithelial sodium channel, playing a role in the humidification of epithelial surfaces. CF typically lowers the life expectancy of those born with it, although new treatments (as discussed herein) are allowing a longer life span for CF patients; but these treatments do have the potential to create some issues with fat-soluble vitamin serum levels with prior art multivitamin formulations based on today's practiced standards of care.

One such new treatment is CFTR modulator formulation (“MF”) therapy. MF therapy partially corrects the malfunctioning CFTR protein and depending on the specific MF therapy and the individual patient, offers varying degrees of correction; but restoration of low-end CFTR function at best. This correction, in addition to improving respiratory function (i.e., FEV1), may improve pancreatic function, reduce fat malabsorption, affect the absorption of fat-soluble vitamins from the gastrointestinal (“GI”) tract, and alter liver vitamin stores of certain fat-soluble vitamins. For example, in one case report of a 12-year-old girl, mildly pancreatic insufficient (“PI”), was prescribed pancreatic enzymes and began taking a multivitamin containing 1,440 IU of total vitamin A, of which 432 mcg was preformed vitamin A. Thirty (30) days later, the patient began ivacaftor, a MF therapy. Thirteen months later, ivacaftor was replaced with elexacaftor/tezacaftor/ivacaftor (“ETI”), a MF therapy with a greater effect on restoration of CFTR function than ivacaftor. At this time her multivitamin was changed, marginally increasing total vitamin A content to 1,553 IU and preformed vitamin A to 436 mcg. Two months after beginning ETI, the ETI and multivitamin were discontinued due to elevated intra-cranial pressure. Serum retinol was measured as 48 ug/dl and was within normal limits. Seven weeks later, despite discontinuation of her multivitamin, serum retinol was found to be mildly elevated (57 ug/dl). Given the intake of diet alone, this elevation and progressive increase in serum retinol cannot be explained and supports the suggestion of a release of vitamin A from liver stores. This patient was heterozygous for F-508del. The increased absorption of fat-soluble vitamins, and in particular preformed vitamin A, have the potential to cause vitamin toxicity with prior art multivitamin formulations that are commonly used in CF care today.

While there is a plurality of mutations in the CFTR Gene that can cause CF, current CFTR modulator therapies are mutation specific. The most common CFTR variant in the U.S. is F508del. Over 85% of individuals in the 2020 Annual CF Foundation Patient Registry who were genotyped had at least one copy of the F508del. Individuals with one copy of this deletion are called heterozygous for F508del. People with CF who have two copies of F508del are called homozygous, and account for about 45% of this 2020 population. Patients with two copies tend to have more severe lung disease, characterized by more frequent lung exacerbations, lower lung function (i.e., FEV1), and lower absorption of fats and fat-soluble vitamins, as well as other disease-related complications. It appears that modulators have a greater effect on restoring low end CFTR function change—whether a potentiator, corrector, or both a potentiator and corrector—in individuals homozygous for F508del, versus heterozygotes for F508del, or another variation in CFTR; and such an effect may be greater when a modulator is started earlier in life. The clinical effectiveness of modulator therapy may be most pronounced in those younger (e.g., 4 years of age, 6 years of age) versus older individuals with CF (e.g., persons in their 30's, 40's and 50's) and their effect on absorption is likely subject to the underlying reason for loss of pancreatic function.

To date, the FDA has approved four (4) CFTR modulators, including Kalydeco® (ivacaftor, a potentiator), Orkambi® (lumacaftor/ivacaftor, a corrector and potentiator combination more potent than Kalydeco), Symdeko® (tezacaftor/ivacaftor, a corrector and potentiator even more potent than both Kalydeco and Orkambi), and Trikafta® (elexacaftor/tezacaftor/ivacaftor, two correctors and one potentiator. Trikafta is the most effective MF therapy in restoring CFTR function approved by FDA to date.

Ivacaftor, the first MF therapy approved by FDA, works by opening the gates of the CFTR protein and helps to keep those gates open longer. Tezacaftor and Elexacaftor help the CFTR protein to form the correct shape, so the CFTR protein can get to the surface of the cell, where the protein is normally located. Early MF therapies, Ivacaftor (Kalydeco), Lumacaftor/Ivacaftor (Orkambi) and

Tezacaftor/Ivacaftor (Symdeko), have been shown to be only moderately effective, resulting in an average of about a 2% increase in lung function (as measured by FEV1). Elaxacaftor/Tezacaftor/Ivacaftor (Trikafta), a triple combination of modulators, and the most recent market introduction by VERTEX, brings many more CFTR gates to the right place in the cell, and increases FEV1 by an average of 12% in patients with CF. This triple MF therapy, with two correctors and one potentiator, allows many more chloride ions to move in and out of the cell and restores low-end function of the CFTR protein to a greater degree that previous MF therapies. It is this increased effectiveness that has brought attention to the subject of the formulation of prior art multivitamins used in the supportive care of CF, and concerns surrounding elevated fat-soluble vitamin serum levels, and the potential for vitamin-related toxicity with the advances and use of MF therapy.

While the majority of interest in CFTR modulator therapy has focused on the airway passages (e.g., the lungs), due to chronic airway obstruction, inflammation and infection—as this results in progressive damage to the lungs and accounts for about 80% of morbidity and mortality in CF—the CFTR Protein is also absent or functionally impaired on intestinal, biliary and pancreatic epithelium in CF, affecting the body's ability to absorb essential nutrients from the GI tract. The PROMISE Study, a prospective, multi-center, observational study of some 180 patients, sought to measure the clinical effectiveness of ETI in children 6-11 years of age, with one or more copies of the F508 deletion. The study included patient-reported outcomes and basic quality-of-life measures: diabetes, bone health, microbiology, lung function, gastrointestinal health, blood sugar and liver function. The protocol included one measurement before ETI therapy and 4 subsequent measurements—at one month, 6 months, 12 months, and 24 months. A reported adverse event with ETI usage was weight gain. While not addressed in the study as a causative factor, weight gain was potentially related to the improvement in the absorption of dietary fats. This increased absorption of fats would also likely result in an increased absorption of the fat-soluble vitamins, an increase that is variable based on the MF therapy prescribed, the underlying cause resulting in the loss of pancreatic function, and the age of the individual. A measure not included in the PROMISE Study was measurement of serum fat-soluble vitamin levels.

Data available to evaluate of the effects of ETI on fat-soluble serum levels has been limited to case reports and small cohorts, and published findings on the effects of early MF therapies are inconsistent surrounding serum fat-soluble vitamin level changes. Of note, the CF Foundation has not amended its Guidelines for fat-soluble vitamin intake in patients prescribed MF therapy, nor cited any concern for patients taking prior art CF multivitamin formulations on MF therapy to date; and has expressed that it is “too early to draw any conclusions” around MF therapy and fat-soluble serum vitamin levels with prior art CF multivitamins. The data disclosed herein suggested otherwise. The unexpected discovery that patients undergoing modulator therapy had altered vitamin levels, including lowered levels of vitamins A, E and D provide an opportunity to improve care for those patients.

About 85% of patients with CF are pancreatic insufficient or suffer from pancreatic insufficiency due to CFTR dysfunction, resulting in obstruction of the pancreatic duct by thick mucus that leads to the destruction of the exocrine pancreatic tissue that produces lipases, proteases and amylases involved in the digestion of food. Once a diagnosis of PI is made, pancreatic enzyme therapy (“PERT”) is often initiated. Despite PERT intervention, malabsorption of fats and fat-soluble vitamins (e.g., A, D, E, K) remains an ongoing manifestation. Historical guidance has indicated that certain multivitamin formulations could be beneficial in the treatment of CF patients, and they are widely used. However, these prior art multivitamins have not been formulated with our consideration of the recent advancements of MF therapy in mind and particularly ETI, and accordingly, there is a need in the prior art CF multivitamins formulations for a change.

As noted, there is a plurality of mutations in the CFTR Gene that can cause CF, however, current MF therapies are mutation specific, and use requires at least one copy of the F508del mutation. In some patients with the F508del mutation, concomitant medical issues (e.g., liver damage, worsening liver function), adverse events (e.g., rash, elevated liver enzymes due to MF's) and individuals who do not meet the age-specific indication for prescribing, limit MF use. For these reasons, there remains a need for prior art CF multivitamins. These multivitamins, however, do present a recognized and serious limitation; discoloration and staining due to the beta-carotene (i.e., pro-vitamin A) component. Early multivitamins that met the CF Foundation Guidelines, and specifically the guideline for vitamin A content, contained only preformed vitamin A as either the palmitate or acetate salt. Preformed vitamin A, however, is insatiably absorbed and absorbed independent of serum vitamin A levels and the need for supplemental vitamin A. One study of patients with CF found that twenty-five percent (25%) did not require any vitamin A supplementation, and individuals were supplemented with vitamin A in a range from 0-20,000 IUs daily. In another study, Graham-Maar concluded “that usual care” of children with CF may result excessive vitamin A intake and possible toxicity that would increase the risk of CF-related liver disease and bone complications. These patients used vitamins with only preformed vitamin A. Maqbool (Journal of Cystic Fibrosis 7 (2008) 137-141) concluded the same; and that “under current patterns of care” 58% of subjects had elevated serum retinol concentrations when using preformed vitamin containing multivitamins that met CF Foundation Guidelines for vitamin A intake.

The changes to address the issues and limitations above with prior art oral multivitamins—namely high serum vitamin A levels, and the discoloration and staining due to beta-carotene (i.e., pro-vitamin A) are provided by the present disclosure.

### DESCRIPTION OF DRAWINGS

To further illustrate the advantages and features of the present disclosure, a more particular description of the invention will be rendered by reference to specific embodiments thereof which are illustrated in the appended images. It is appreciated that these images are not to be considered limiting in scope. The inventions will be described with additional detail through the use of the accompanying images in which:

FIGS. 1A and 1B show one embodiment of the prior art multivitamins staining on different fabric swatches after individual treatments with peroxide and bleach.

FIGS. 2A and 2B show one embodiment of the multivitamin of the present disclosure not leaving stains on different fabric swatches after individual treatments with peroxide and bleach.

FIG. 3 shows the color of one embodiment of the color of the multivitamin disclosed herein which is not the typical orange/red color but a cream color due to the presence of the amended beta-carotene.

FIGS. 4A and 4B show one embodiment of the multivitamin of the present disclosure not leaving stains on different fabric swatches after individual treatment with a 3% hydrogen peroxide solution.

### DETAILED DESCRIPTION

In one embodiment of the current invention, the beta-carotene component of the disclosed multivitamin is formulated to be non-discoloring, whereas prior art beta-carotene containing liquid multivitamin supplements discolor and often permanently stain certain materials (e.g., clothing, bibs, dish towels, carpeting, rugs), and discolors certain other areas and stains where the beta-carotene component contacts (e.g., teeth, tongue, hands).

In another aspect of this invention, the present disclosure provides a multivitamin specifically formulated to be used in patients undergoing CFTR modulator therapy (e.g., Trikafta®, Orkambi®, Kalydeco®). The multivitamin includes, in various embodiments, an amended, non-staining pro-vitamin A (non-staining beta-carotene), pro-vitamin A, or both, preformed vitamin A, vitamin E, vitamin K, a solubilizer, one or more emulsifiers, a bile fluid secretion stimulator, high dose vitamin C and a probiotic formulation. Also, to be considered within the scope of this embodiment is a multivitamin containing at least two of amended, non-staining pro-vitamin A (amended, non-staining beta-carotene), pro-vitamin A, or both, preformed vitamin A, vitamin E, vitamin K, a solubilizer, one or more emulsifiers, a bile fluid secretion stimulator, high dose vitamin C and a probiotic formulation. Also, to be considered within the scope of this embodiment is a multivitamin containing at least three of amended, non-staining pro-vitamin A (amended, non-staining beta-carotene), pro-vitamin A, or both, preformed vitamin A, vitamin E, vitamin K, a solubilizer, one or more emulsifiers, a bile fluid secretion stimulator, high dose vitamin C and a probiotic formulation.

Also, to be considered within the scope of this embodiment is a multivitamin containing at least the amended, non-staining pro-vitamin A (amended, non-staining beta-carotene) and two of preformed vitamin A, vitamin E, vitamin K, a solubilizer, an emulsifier, a bile fluid secretion stimulator, high dose vitamin C and a probiotic formulation.

Also, to be considered within the scope of this embodiment is a liquid vitamin containing only amended, non-staining pro-vitamin A (amended, non-staining beta-carotene).

CF causes PI and reduces intestinal absorption of fats and fat-soluble vitamins such as vitamins A, D, E, and K. PI is present in up to 85% of CF patients at birth. CFTR Modulator Therapy has been reported to improve FEV1 in the range of 2% to 12% depending on the MF. Qualitatively, this change in the CF lung due to CFTR function is also seen in the GI tract. It is not known whether this change in the GI tract is comparable to the 2% to 12% and at the lower range of CFTR function, as seen in the CF lung.

One aspect of this invention in one embodiment is to decrease the preformed vitamin A content in the formulation of a multivitamin from what is taught in the prior art, and to increase pro-vitamin A content. Pro-vitamin A may be the novel, amended, non-staining beta-carotene or beta-carotene in the form as found in prior art beta-carotene containing multivitamins, or both.

Vitamin A is an organic molecule that is an essential micronutrient which an organism needs in small quantities for the proper functioning of overall metabolism. Vitamin A, a fat-soluble vitamin, acts as a regulator of cell and tissue growth and cell differentiation and is an essential nutrient. Vitamin A is a group of compounds that includes retinol, retinal, and retinoic acid, all preformed vitamin A; and several pro-vitamin A carotenoids, most notably beta-carotene.

Preformed vitamin A, however, is known to be insatiably absorbed and increases the propensity for vitamin A toxicity (e.g., hypervitaminosis A), and is absorbed whether needed by the body, or not. In this embodiment, the amount of pro-vitamin A per serving for persons over three (3) years of age is increased to a total vitamin A content of 6000 mcg, with preformed vitamin A reduced to 3.5% to and approximately 210 mcg to 330 mcg of total vitamin A content. Currently marketed prior art multivitamin formulations used in CF care range from 8% and 436 mcg to 12% and 576 mcg of preformed vitamin A. For newborns and infants up to 3 years of age, total vitamin A content is increased to 2000 mcg per 3 ml or 4 ml of volume serving size, with preformed vitamin A content reduced to 7% to 8% of the total vitamin A content and to approximately 140 mcg to 160 mcg respectively. Current prior art formulations include preformed vitamin A in a range from 13% and 225 mcg to 25% and 347 mcg per serving. The increase in pro-vitamin A over prior art formulations is anticipated to accommodate for individuals with genetic variation in the BCMO1/BCO1 gene, a variation reported to occur in 25-47% of the general population. These individuals are called “poor converters” of pro-vitamin A. A single T substitution in the rs7501331 results in a conversion decrease of 32%; substitution with a T allele on both rs12934922 and rs7501331 results in an even more inefficient conversion of pro-vitamin A and a 69% decrease in conversion efficiency. In an alternate embodiment, total vitamin A is not increased, but preformed vitamin A remains between approximately 210 mcg and 336 mcg per serving for those over three (3) years of age, and approximately 140 mcg to 160 mcg, formulated to 3 ml or 4 ml in volume per serving, for newborns and infants up to three (3) years of age.

The various forms of vitamin A, when absorbed, are solubilized into micelles in the intestinal lumen and absorbed by duodenal mucosa. Preformed vitamin A (e.g., retinyl acetate, retinyl palmitate) is absorbed from the digestive tract via passive diffusion, and insatiably absorbed. Absorption is independent of the body's need for any supplemental vitamin A. Pro-vitamin A is taken up by enterocytes by the membrane transporter protein scavenger receptor B1 (SCARB1), which is upregulated in times of vitamin A deficiency. If vitamin A status is within the normal range, SCARB1 is downregulated, reducing the conversion and absorption of pro-vitamin A. Also downregulated is the enzyme beta-carotene 15, 15′ monooxygenase, coded for by the BCMO1 gene, responsible for cleaving beta-carotene to retinal.

Overconsumption of preformed vitamin A can be highly toxic and is especially contraindicated prior to and during pregnancy, as it can result in severe birth defects. The UL for preformed vitamin A in adults is 3000 mcg per day. There is no daily UL for pro-vitamin A as beta-carotene.

While multivitamin supplement formulations that contain beta-carotene to lower preformed vitamin A content offer clear safety advantages to individuals, beta-carotene does present a challenge—staining. In a liquid form, prior art beta-carotene stains clothing when it comes in contact with the material. The stain is almost always impossible to remove. When beta-carotene used in a chewable multivitamin, prior art beta-carotene is often reported as staining the teeth or discoloring the tongue or oral epithelia. This novel, amended, non-staining beta-carotene, also addresses these discoloring and staining issues with chewable forms and any form that dissolves, releases or disperses beta-carotene within the oral cavity.

Early multivitamins used in CF care contained only preformed vitamin A. A study was conducted by Brei (Clin Nutr. 2013 October; 32(5): 805-10) to assess vitamin status and appropriate supplementation in patients receiving vitamin A. The Brei study found (a) that the range of the supplementation dosage was from 0-20,000 IU/day, (b) that 25% of patients did not need any vitamin A supplementation, and (c) total vitamin A intake exceeded the recommended daily UL in 69% of studied subjects. This, and other findings, including Graham-Maar (Am J Clin Nutr. 2006 July; 84(1): 174-82), led to multivitamin formulations that included both pro-vitamin A and preformed vitamin A to minimize this potential for toxicity. The prior art pro-vitamin, however, introduced this new issue—staining by the beta-carotene.

Vitamin A is obtained from the diet as either preformed vitamin A or as pro-vitamin A carotenoids. Total vitamin A intake in the US averages approximately 600 mcg per day, and of this, about 70-75% is thought to be due to the intake of preformed vitamin A in eggs, meat, butter, milk, and fortified foods. The recommended daily allowance of vitamin A is (i) 900 mcg for men 14 and older and (ii) 700 mcg for women 14 and older, (with a UL of 2000 mcg for ages 14-18 and 3000 mcg for those over 18 years of age), (iii) 9-13 years of age, 600 mcg, (with a UL of 1700 mcg), (iv) 400 mcg for children 4-8 years, (with a UL of 900 mcg), (v) 300 mcg for 1-3 years of age, (with a UL of 600 mcg), (vi) 500 mcg for ages 7-12 months, (with a UL of 600 mcg), and (vii) 400 mcg for 0-6 months, (with a UL of 600 mcg). CF prior art multivitamins often contain significantly higher amounts of preformed vitamin A than the age-specific, daily RDA's, independent of amounts of preformed vitamin A from dietary sources. While MF therapy helps to restore low end CFTR function, the variability of restoration of CFTR function is based on the choice of MF, varied individual patient response due to underlying cause of loss of pancreatic function, differences in dietary intake of preformed vitamin A in patients, coupled with the knowledge that beta-carotene is only converted to an active form of vitamin A as needed, one aspect of this invention introduces a significant change from prior art formulations in both total vitamin A per serving content and the percentage of preformed and pro-vitamin A forms to overcome risks of toxicity in patients receiving MF therapy and a prior art CF multivitamin.

Additionally, vitamin E plasma levels are likely affected by CFTR modulator therapy. One potential cause of this reduction may be the induction of cytochrome P450 system; another may be an increase in bile production or release. A review of studies with early MF therapies shows inconsistent results in the changes seen in vitamin E levels in patients with CF:
Study #1: No significant change in serum vitamin E levels. Patients were heterozygous for F508del. MF therapy was ivacaftor. Duration: 3 months.
Study #2: Significant decrease in serum vitamin E levels at year one and year two with lumacaftor/ivacaftor. All patients were homozygous F508del.
Study #3: Increased vitamin E levels, a change positively correlated with treatment months. Treatment ranged from 9 to 29 months. 27 patients were homozygous F508del; 28 patients were heterozygous F508del. Before MF therapy with lumacaftor/ivacaftor, homozygous F508del had significantly lower vitamin E serum levels than heterozygous F508del.

While no publicly available data is available on the effects of ETI on vitamin E serum levels, we believe it is likely that:
1. Changes in CFTR function with MF therapies are not isolated to the lung. These changes are also realized—to some and varying degrees—in intestinal, biliary, and pancreatic epithelium.
2. The restorative function of the CFTR protein when ETI is prescribed, versus earlier and other MF therapies, affects a greater number of patients, and typically increases vitamin E levels in both individuals heterozygous and homozygous for F508del; a finding that appears to differ than when lumacaftor/ivacaftor or ivacaftor are prescribed, where the effects observed include no change, increases and decreases in vitamin E serum levels.

Accordingly, another aspect of this invention, in one embodiment, is to decrease the amount of vitamin E in the multivitamin to approximately 75 mg-85 mg (112 IU-127 IU) per serving for those over 3 years of age; recognizing that “the degree of effect” of MF therapy on vitamin E serums levels is a function of potency of the MF therapy, the homozygous versus heterozygous F508del status of the individual, and potential duration of MF therapy. For newborns and infants up to 3 years of age, vitamin E is reduced by 50%, from 50 IU-100 IU (33.5 mg-67 mg) per age-based serving in prior art multivitamins, to 25 IU-50 IU (16.75 mg-33.5 mg) per age-based serving.

Current prior art CF-specific multivitamins for those over 3 years of age include 101 mg (150 IU) to 134 mg (200 IU) of vitamin E per serving in their formulations. Of note, additional vitamin E is included as mixed tocopherols or a tocopherol rich extract, referenced in the Other Ingredients sections of some labels, but not included in the total vitamin E content per serving in the labels of these prior art multivitamins. This serves as an additional source and amount of Vitamin E, despite the Supplement Facts label disclosure of just 150-200 IUs per serving for those over 3 years of age. In one embodiment, the amount of vitamin E may be between about 75 mg-85 mg (112 IU-127 IU) per serving for those over 3 years of age. For newborns and infants, an amount of 16.75 mg-33.5 mg (25 IU-50 IU) per serving, and a 50% reduction in vitamin E content per age-based serving when compared to prior art formulations. These changes, a decrease in vitamin E content, is inconsistent with changes over the last two decades, where multivitamin preparations used in CF have increased vitamin E content. A decrease would only be contemplated by one who considers the effects of MF therapy on serum vitamin levels and the effects of ETI, in particular. While it is anticipated that individuals who are homozygous F508del will realize greater increases in serum vitamin E levels when prescribed MF therapy, it is also believed that with a shift in prescribing to ETI—the most potent modulator approved by FDA to date and commercially available—this reduced vitamin E content will provide the needed therapeutic serum levels for both individuals homozygous and heterozygous for F508del receiving MF therapy.

While prior art multivitamins, over the last two decades, have increased the amount of vitamin K per serving, vitamin K status remains low in about 1 out of 5 CF patients. CF Bones, a study conducted from 2000-2002, found that fifty percent (50%) of the CF patients who were PI reported low vitamin K status. The adult daily recommended intake of vitamin K for adults by the CF Foundation was 300 mcg-500 mcg. CF Avanti, a later study from 2007-2010, found that about one in five patients (19%) still had low vitamin K status, despite more than doubling vitamin K intake, despite the effects of MF on vitamin A and vitamin E. Another embodiment of this invention is to increase vitamin K serving size to 600 mcg for children up to 12 months and 1,200 mcg for children 1-3 years of age; and 1,200 mcg per serving for children four (4) to eight (8) years of age, and 1,200 mcg per serving for individuals 9 years of age and older.

The vitamin of the present disclosure, in one embodiment, also includes a solubilizer. While the prior art CF multivitamins often contain emulsifiers, they do not contain solubilizers. An understanding of the difference between an emulsifier and a solubilizer is important to the understanding of this invention.

Emulsifiers enable the suspension of oil in water; they are not water soluble. This results in large oil droplets suspended in an aqueous medium, like fats and fat-soluble vitamins within the contents in the small intestine. Emulsification of fats is a critical part of digestion and promotes micelle formation in the presence of bile salts. Most of the fluid in the digestive tract is water-based, so when one consumes fat, digested fat tends to form large globules in the intestine. Large globules are incompletely and inefficiently absorbed and limit the absorption of fat-soluble vitamins. In individuals without CF, emulsification is promoted by bile; in CF, however, bile composition differs from individuals without CF. The bile pool is typically reduced due to CFTR function in the gall bladder, and the complete or partial obstruction of the bile duct that can occur. This inefficiency may explain why a significantly higher intake of fat-soluble vitamins (A, D, E, K) has been required in patients with CF, than individuals without CF; and despite these higher intakes, why many CF patients remain low fat-soluble vitamin status, despite ongoing increases in fat-soluble vitamin intake over the last 20+ years; and why PERT fails to fully normalize fat digestion and absorption.

Solubilizers, on the other hand, help to make otherwise insoluble materials soluble in an aqueous medium, like fats and fat-soluble vitamins in water. Solubilization enhances the solubility of fatty acids by several magnitudes (100 to 1000-fold) and absorption is achieved by the formation of mixed micelles. The diameter of emulsified lipid droplets ranges from 100 to 1000 nm, whereas the diameter of mixed micelles ranges from 3 to 5 nm. Solubilization is necessary for hydrophobic components to efficiently overcome the diffusion barrier of the unstirred water layer of the cells lining the small intestines (i.e., enterocytes), which separate the enterocytes from the luminal contents of the intestine, and to more efficiently and more completely absorb fats and support the absorption of fat-soluble vitamins—notably the products from long chain fatty acids.

Accordingly, the multivitamin in the example in Exhibit I includes a solubilizer. In one embodiment, the solubilizer is a water soluble solubilizer. In one preferred embodiment, the water soluble solubilizer are cyclodextrins (e.g., alpha-cyclodextrin, beta-cyclodextrin, beta-cyclodextrin derivatives) or polyoxymethylene (20) sorbitan monolaurate (“Polysorbate 20”). In an alternative embodiment, the solubilizer is a water miscible solubilizer such as glycols, which include glycerin USP, propylene glycol, and polyethylene glycol “PEG”.

The multivitamin of the present disclosure also includes a bile fluid secretion stimulator, such as peppermint oil (“PO”) in one embodiment. Bile acids, the essential components of bile, are involved in some critical physiological functions such as intestinal digestion and supporting lipid absorption. Therefore, they contribute to digestive function and the prevention of disease by promoting bile secretion and raising bile acid content. PO a natural source from vegetable oil that mainly contains L-menthol, L-menthone and isomenthone, stimulates bile fluid secretion and thus has a choleretic effect. This effect may be the result of upregulating CYP7A1 and FXR mRNA levels, suggesting that the molecular mechanisms are related to gene expression involved in bile acid synthesis.

In addition, while PO may be used as a flavoring (i.e., to taste mask) in foods and beverages, no prior art CF-specific multivitamins include PO to stimulate bile production. The presence of bile salts is required to support the formation of micelles and facilitate the absorption of fats and fat-soluble vitamins and is supported by the addition of PO into the multivitamin matrix.

The multivitamin of the present disclosure also includes high dose vitamin C in one embodiment in the range of approximately 350 mg to 1200 mg daily which may serve to induce the opening of the CFTR Chloride channels and enhance epithelial secretions to reduce the stickiness of the mucus on intestinal epithelia in the gut. Prior art multivitamins for those over 3 years of age include 70-100 mg of vitamin C per serving for individuals over 3 years of age, and 45 mg per serving for newborns to 12 months and 90 mg per serving for those 1-3 years of age. This novel formulation developed for those on MF therapies, significantly increases vitamin C content with the design to increase the time of opening or delay the closing of CFTR channels and aid in the support of absorption. In a preferred embodiment, vitamin C is in a liposomal form. Alternatively, in another embodiment, vitamin C may be from ascorbic acid or a combination of ascorbic acid and zinc ascorbate. The recommended daily allowance (“RDA”) for vitamin C, in those 19 years of age and older is 90 mg daily for men and 75 mg for women. The UL for adults is 2000 mg. For (i) 14-18 years of age, the RDA is 75 mg and 65 mg for males and females respectively (1800 mg UL); (ii) 45 mg for individuals 9-13 (1200 mg UL); (iii) children 4-8 years of age 25 mg (650 mg UL); and (iv) children 1-3, 15 mg (400 mg UL). There is no established UL for newborns and infants from 0 to 12 months.

Vitamin C is a biological regulator of CFTR-mediated Cl secretion in the epithelia. While focus has previously been directed to the nasal epithelium, vitamin C can theoretically activate Cl transport in the GI tract at appropriate concentrations. At the appropriate concentrations, vitamin C induces the openings of the CFTR Chloride channels—increasing its average open probability from zero to 0.21 (+0.08)—enhancing epithelial secretions and reducing the stickiness and viscosity of mucus on the surface of the intestinal epithelia. This concept is further supported by the fact that high doses of vitamin C can cause secretory diarrhea as a side effect, which is readily explained by its stimulatory effect on CFTR-mediated Cl secretion in intestinal epithelial cells. Vitamin C crosses the small intestine, and then enters the plasma. We suggest that levels of vitamin C of from 150 mg to 200 mg per serving in newborns and infants, and from 400 mg to 600 mg per serving in those 3 years of age, that when taken orally as a single dose, can exert influence over CFTR Cl channels and potentially support the uptake of fat-soluble vitamins (A, D, E, K) by inducing the opening of CFTR Cl channels of epithelial cells bordering the CF small intestine, without a detectable increase in cAMP.

The multivitamin disclosed herein optionally includes zinc as well. In one embodiment for those over 3 years of age the multivitamin contains about 10 mg zinc per serving with the zinc being from either zinc oxide or zinc ascorbate. In another embodiment for those under 3 years of age, the multivitamin contains about 5 mg zinc as zinc oxide.

CF patients often suffer from abnormal gut flora. Accordingly, in one embodiment, the multivitamin of the present disclosure provides a probiotic formulation to help correct this abnormality. In one embodiment, the probiotic formulation includes at least one of Lactobacillus rhamnosus GG, Lactobacillus reuteri, Lactobacillus rhamnosus and Bifidobacterium animalis. In an alternate embodiment, the multivitamin of the present disclosure includes at least two (2) of the forgoing.

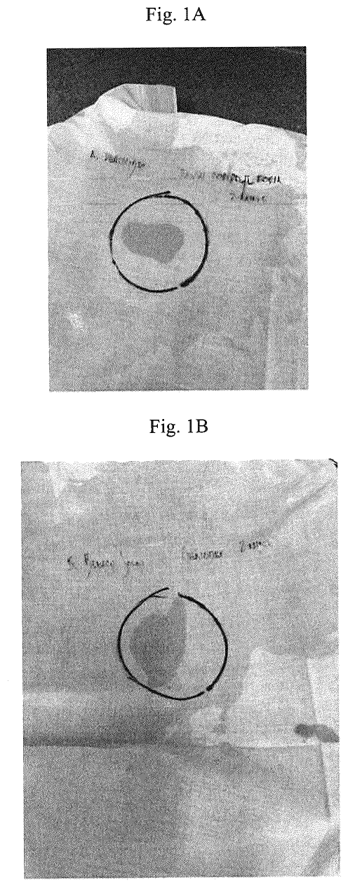

Now referring to FIGS. 1A through 4B, the multivitamin of the present disclosure containing an amended beta-carotene does not stain surfaces (including without limitation, clothing, fabrics, skin, teeth, tongue, etc.) as the prior art vitamins do. For example, FIGS. 1A and 1B show a prior art multivitamins staining on different fabric swatches after individual treatments with peroxide and bleach. Such staining is essentially permanent. This staining is known to occur no matter the form of vitamin, liquid, chewable, gummie, soft gel or the like. For example, chewing a prior art multivitamin chewable tablet results in a physical change—smaller and small pieces of the original chewable tablet dosage form—so that the tablet may be swallowed. These pieces and particles, suspended in the salivary fluid, can discolor the teeth and tongue when in contact with an aspect of the oral cavity or teeth. This is particularly problematic for the administration of a chewable tablet to those who have braces to straighten the teeth, or any other oral apparatus where particles may come to adhere. The multivitamin of the present disclosure solves this problem.

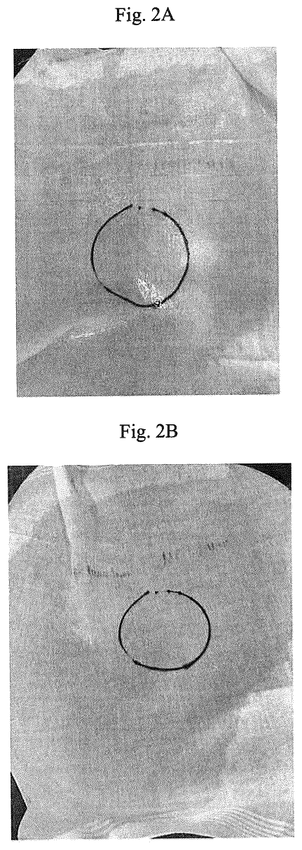

For comparison, FIGS. 2A and 2B show one embodiment of the multivitamin of the present disclosure not staining different fabric swatches after individual treatments with peroxide and bleach. The same is true of skin and teeth, the multivitamin of the present disclosure does not stain as the prior art vitamins do.

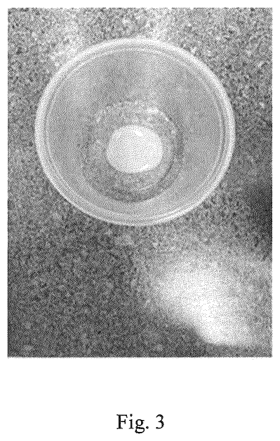

FIG. 3 shows the color of one embodiment of the color of the multivitamin disclosed herein which is not the typical orange/red color but rather a cream color due to the presence of the amended beta-carotene.

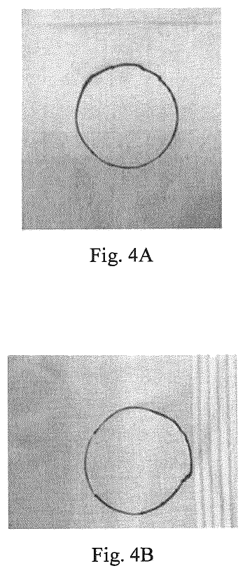

FIGS. 4A and 4B show a different embodiment of the multivitamin of the present disclosure not leaving stains on different fabric swatches after individual treatment with a 3% hydrogen peroxide solution.

The multivitamin of the present disclosure can be formulated for human use and may comprise and may, optionally include a pharmaceutically acceptable carrier. Additionally, methods of treatment of patients in need of treatment or in need of prevention of the side-effects of modulator therapy, such as those discussed herein, are within the scope of this disclosure.

The term “individual” or “patient” as used herein refers to any animal, including mammals, such as, but not limited to, mice, rats, other rodents, rabbits, dogs, cats, swine, cattle, sheep, horses, primates, or humans.

The term “in need of prevention” as used herein refers to a judgment made by a caregiver that a patient requires or will benefit from prevention. This judgment is made based on a variety of factors that are in the realm of a caregiver's expertise and may include the knowledge that the patient may become ill as the result of a disease state that is treatable by a compound or pharmaceutical composition of the disclosure.

The term “in need of treatment” as used herein refers to a judgment made by a caregiver that a patient requires or will benefit from treatment. This judgment is made based on a variety of factors that are in the realm of a caregiver's expertise and may include the knowledge that the patient is ill as the result of a disease state that is treatable by a compound or pharmaceutical composition of the disclosure.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or value beyond those recited. Headings, lists, and numbering included herein are for ease of explanation only and are not meant to be limiting.

The terms “about” and “approximately” shall generally mean an acceptable degree of error or variation for the quantity measured given the nature or precision of the measurements. Typical, exemplary degrees of error or variation are within 20 percent (%), preferably within 10%, more preferably within 5%, and still more preferably within 1% of a given value or range of values. Numerical quantities given in this description are approximate unless stated otherwise, meaning that the term “about” or “approximately” can be inferred when not expressly stated. As used herein, the singular forms “a”, “an” and “the” are intended to include the plural forms as well, unless the context clearly indicates otherwise.

1. A multivitamin comprising beta-carotene, wherein the beta-carotene is non-discoloring.

2. The multivitamin of claim 1, further comprising preformed vitamin A.

3. The multi-vitamin of claim 1, wherein the multivitamin further comprises at least one of vitamin E, vitamin K, a solubilizer, one or more emulsifiers, a bile fluid secretion stimulator, high dose vitamin C and a probiotic formulation.

4. The multi-vitamin of claim 3, wherein the multivitamin further comprises at least two of vitamin E, vitamin K, a solubilizer, one or more emulsifiers, a bile fluid secretion stimulator, high dose vitamin C and a probiotic formulation.

5. The multivitamin of claim 1, wherein the multivitamin is liquid.

6. The multi-vitamin of claim 5, wherein the multivitamin further comprises at least one of vitamin E, vitamin K, a solubilizer, one or more emulsifiers, a bile fluid secretion stimulator, high dose vitamin C and a probiotic formulation.

7. The multi-vitamin of claim 5, wherein the multivitamin further comprises at least two of vitamin E, vitamin K, a solubilizer, one or more emulsifiers, a bile fluid secretion stimulator, high dose vitamin C and a probiotic formulation.

8. The multivitamin of claim 7, wherein the multivitamin is liquid.

9. The multivitamin of claim 3, wherein the solubilizer is a water soluble solubilizer.

10. The multivitamin of claim 4, wherein the solubilizer is a water miscible solubilizer.

11. The multivitamin of claim 1, further comprising a bile fluid secretion stimulator.

12. The multivitamin of claim 11, wherein the bile fluid secretion stimulator is peppermint oil.

13. A multivitamin for a patient over three (3) years of age, the multivitamin comprising:
a. vitamin A
b. vitamin E; and
c. a solubilizer.

14. The multivitamin of claim 13, wherein the solubilizer is a water soluble solubilizer.

15. The multivitamin of claim 14, wherein the solubilizer is a water miscible solubilizer.

16. The multivitamin of claim 15, further comprising a bile fluid secretion stimulator.

17. The multivitamin of claim 16, wherein the bile fluid secretion stimulator is peppermint oil.

18. The multivitamin of claim 17, further comprising at least two (2) of vitamin C, vitamin K and zinc.

19. A multivitamin comprising:
a. preformed vitamin A;
b. pro-vitamin A; and
c. a solubilizer.
